(19) United States
(12) Patent Application Publication
(10) Pub. No.: US 2022/0099692 A1
(43) Pub. Date: Mar. 31, 2022

(54) AUTOMATED LIQUID HANDLING SYSTEM AND METHOD FOR DEPOSITING BIOLOGICAL SAMPLES FOR MICROSCOPIC EXAMINATION

(71) Applicant: YANTAI AUSBIO LABORATORIES CO., LTD., Yentai, Shandong (CN)

(72) Inventor: Zhaoqiang WANG, Yantai (CN)

(21) Appl. No.: 17/423,632

(22) PCT Filed: Jan. 16, 2019
(86) PCT No.: PCT/CN2019/071992

§ 371 Date: Jul. 16, 2021

(51) Int. Cl. G01N 35/10; B01L 9/00; B01L 3/00; G01N 35/00; G01N 1/30; C12M 1/32; C12M 1/00; C12M 3/00; C12M 1/36

(52) U.S. Cl. CPC G01N 35/10; B01L 2300/08; B01L 9/523; B01L 3/56; B01L 3/50855; G01N 35/0099; G01N 1/30; C12M 23/12; C12M 23/22; C12M 23/48; C12M 23/50; C12M 41/48; G01N 2035/1051; B01L 2300/168; B01L 2300/123; G01N 35/1065

(57) ABSTRACT

Automated liquid handling system for processing a plurality of samples in at least one microscope sample carrier, wherein the microscope sample carrier comprises a plurality of sample deposition wells, wherein each sample deposition well is defined on its lateral sides by one or more lateral walls and on its bottom side by a sample deposition surface,
the automated liquid handling system comprising:
   a centrifuge adapted to centrifuge the microscope sample carrier;
   an automated transportation device
      adapted to transfer the plurality of samples and/or a plurality of liquids into and/or out of each of the plurality of sample deposition wells of the microscope sample carrier,
      and adapted for transporting the microscope sample carrier across the automated liquid handling system, wherein the automated transportation device is configured to couple with a coupling section of the microscope sample carrier;
one or more storage containers for receiving and/or storing the plurality of samples and/or the plurality of liquids.

### 1. TECHNICAL FIELD

The present invention relates to an automated liquid handling system for processing a plurality of samples in at least one microscope sample carrier, and a method carried out by an automated liquid handling system for such processing of a plurality of first portions of a plurality of biological samples, which are deposited onto a microscope sample carrier. The automated liquid handling system and method according to the present invention are suitable for high-throughput microscopic analysis, in particular in the field of cytological analysis.

### 2. BACKGROUND

Cytological diagnosis is used in a variety of branches in medicine. It refers to the analysis of the structure, function and formation of individual cells of a patient, which allows to derive the physiological condition of the patient and to diagnose various diseases or disease progression.

For a cytological analysis, a sample from the body fluid of the patient, e.g. blood, saliva, urine, epithelial smears, or semen, is obtained and deposited onto a glass microscope slide for examination. Preferably, the sample is evenly distributed on the microscope slide, such that the structure of each individual cell of the sample can be accurately analyzed. An even sample distribution is also critical for the effective implementation of automated microscope diagnostics. Microscope systems usually employ a computer-driven stage under full or interactive user control to scan the microscope slide surface in a pre-programmed manner. In consequence, the boundaries of the region(s) of interest should be clearly defined and restricted to practical dimensions commensurate with the optics technology and time available for microscopic analysis.

Several implementations of sample deposition for cytological analysis are known in the prior art.

For example, smear preparation techniques are frequently used to manually deposit a sample onto a microscope carrier. Such manual smear techniques are fairly inexpensive, but require a certain amount of ability and skill of the practitioner. Moreover, an even distribution of the cells and cell types is difficult to obtain. In particular large cells, such as monocytes, other large leukocytes, or any abnormally large other cells, such as cancer cells, tend to be drawn to the end of the smear, i.e. to the feathered edge, and therefore may be unintentionally excluded from the microscopic analysis.

As an alternative to manual smear preparations, cytocentrifuges are commonly used to deposit biological cell samples onto microscope slides. Here, a small amount of liquid sample is placed onto a microscope slide or another sample receiving surface, which is subjected to a centrifugation step. The centrifugal force acting on the sample throws off excess liquid and spreads the sample radially, forming a thin-layer which covers the sample area on the slide. Such processing allows that cell clumps in the sample can be partially disaggregated and that thin sample layers are formed with minimal adventitious cell overlap. However, due to the centrifugal force acting on the slide surface, the liquid sample is spread in all directions. Therefore, cytocentrifuge devices require the incorporation of waste capturing means such as filter papers, vacuum pumps or wells. In consequence, the construction of cytocentrifuges is complex and expensive. Furthermore, an inherent problem of cytocentrifuges is the use of one microscope slide for each sample. Even with multiple deposition sites on one slide, for example by implementing physical barriers with the slide, only one sample can be applied per centrifugation step. Moreover, undesirable disparities and discrepancies between slides prepared from otherwise identical samples can occur due to sedimentation of the sample during loading into the cytocentrifuge sample chambers. Therefore, care must be taken to minimize the time taken to load the centrifuge sample chambers with samples prior to centrifuging, as well as the sample volume and the surface area covered by the discharge opening. Adversely, sample size limitations of cytocentrifuges can limit the cellular concentrations which may be detected. Therefore, the accuracy of cytocentrifuges may be insufficient for certain cytological analyses, e.g. for the detection of rare cells.

Sample monolayer printing methods are also described in the prior art. For example, US 2011/0070606 describes a system for analyzing cells from body fluids, comprising an applicator for dispensing a fluid comprising body fluid containing cells, said applicator comprising an applicator controller and a tip for dispensing the fluid onto a slide; said tip having a position above the slide.

Further, US 2016/0202278 describes a method for processing a plurality of cell suspensions.

Despite the above described technical advances regarding methods of preparing a microscope slide for sample deposition, the design of the microscope slide itself has not changed fundamentally. Usually, microscope slides are made from glass, and thus are fragile sample carriers. Therefore, all of the above described preparatory methods and devices for the deposition of samples need to be specifically designed to prevent breakage of the microscope slide upon subjection of physical forces, such as during dispensing the sample, centrifugation or other handling routines. In addition, glass slides do not easily allow for high-throughput applications, as the automated movement of multiple slides across various preparation modules, i.e. deposition, drying, fixation, staining, rinsing, reaction and imaging processing, is difficult to implement. Typical processing steps include changing of the stains and solvents, customarily by sequentially lifting one or more slides out of one vessel of treating agent and lowering them into a different vessel of treating agent. Such processing may result in the loss of non-adhered cells to the microscope slide, or even in the contamination of the treating solutions and ultimately other microscope slides.

A variation of a microscope slide has been proposed in U.S. Pat. No. 4,722,598. This document describes a diagnostic microscope slide comprising a plurality of sample wells. The slide is adapted to be used with an automated microscope stage. Such microscope designs allow the deposition of multiple samples in a single slide, but do not facilitate the above described processing steps required for preparing the samples.

Therefore, there still exists a need for an improved method and system to increase the efficiency and accuracy of preparation of slides for microscopic examination, particularly where the microscopic sample is a biological cell-containing liquid. Ideally, such method and system should allow the processing of samples in an automated, parallelized manner to enable high-throughput preparation and microscopic analysis.

### 3. SUMMARY OF THE INVENTION

In a first aspect of the present invention, the above described problems are at least partially solved by an automated liquid handling system for processing a plurality of samples in at least one microscope sample carrier, wherein the microscope sample carrier comprises a plurality of sample deposition wells, wherein each sample deposition well is defined on its lateral sides by one or more lateral walls and on its bottom side by a sample deposition surface, the automated liquid handling system comprising:
a centrifuge adapted to centrifuge the microscope sample carrier;
an automated transportation device
   adapted to transfer the plurality of samples and/or a plurality of liquids into and/or out of each of the plurality of sample deposition wells of the microscope sample carrier,
   and adapted to transport the microscope sample carrier across the automated liquid handling system, wherein the automated transportation device is configured to couple with a coupling section of the microscope sample carrier;
one or more storage containers for receiving and/or storing the plurality of samples and/or the plurality of liquids.

The automated liquid handling system according to the present invention allows the processing of a plurality of samples within the microscope sample carrier for subsequent microscopic examination, comprising processing steps such as sample depositing, staining and washing. The sample deposition surfaces of the microscope sample carrier are physically separated from each other, thereby preventing any cross-contamination of samples during deposition. Suitable samples can be, in particular, biological samples, such as suspensions of biological cells. In this case, the sample deposition surfaces are adapted to hold a first portion of the sample, such as the biological cells of the suspension. The first portions of a sample being deposited on the sample deposition surfaces can be analyzed microscopically, for example using a light or fluorescence microscope, in particular in inverted-mode configuration.

The automated liquid handling system may further comprise a first mounting device adapted to hold the microscope sample carrier for the transfer of the plurality of samples and/or plurality of liquids into and/or out of each of the plurality of sample deposition wells by the automated transportation device. The mounting device serves to stably hold the one or more microscope sample carriers during processing of the samples. The first mounting device may be in particular adapted to hold a plurality of microscope sample carriers in parallel. Such arrangement allows for a high-throughput processing of the samples to be deposited in the microscope sample carriers.

The automated liquid handling system may further comprise a second mounting device adapted to hold the one or more microscope sample carriers for examination of the plurality of samples for examination under a microscope. The one or more microscope sample carriers can be transported to the second mounting device by means of the automated transportation device. The provision of a second mounting device allows that the specific requirements for microscopic analysis, such as adaptation to the geometry of the optical path of the microscope, are fulfilled. In particular, the second mounting device may be adapted to comprise means for holding the microscope sample carriers, such that the sample deposition surfaces of the microscope sample carriers are not covered by the second mounting device. This allows for light microscopic analysis of samples deposited onto the sample deposition surfaces. For example, only the lateral walls, parts thereof, or the top surface of the microscope sample carrier may be held or fixed by the second mounting device.

The automated liquid handling system may further comprise a motorized microscope stage for holding the second mounting device during microscopic examination. Importantly, the second mounting being able to hold one or more microscope sample carriers can thus be controlled by the motorized microscope stage. Thereby, for high-throughput parallel processing, only one motorized stage and control thereof is required for properly positioning the microscope sample carriers in the optical paths of all microscopes of the automated liquid handling system. The motorized stage allows for a correct adjustment of the one or more microscope sample carriers and in particular of the samples, such as biological cells, which are deposited onto the sample deposition surfaces in the focal plane of the microscope. Moreover, the motorized stage allows for the sequential positioning of each well of the one or more microscope sample carriers within the field of view of the microscope. The motorized stage also allows for the precise positioning of the field of view within each well. In general, by precisely moving the well, e.g. by 161 μm to 923 μm in the x and/or y direction, multiple fields of view in the xy-plane of each well can be scanned. This is in particular important for large magnification objectives, e.g. at 1000× magnification, such that the overall surface of each well can be imaged sequentially. Thereby, a stepwise automated imaging of the samples provided in the wells can be performed.

The automated liquid handling system may further comprise an image processing unit. The image processing unit may comprise one or more camera imaging devices, for example one or more CCD, EMCCD or CMOS cameras, each camera coupled into the optical path of one microscope. Alternatively, also two cameras can be coupled to a microscope, for example in case a dual-color image is to be obtained. The camera(s) is (are) used to acquire images or image sequences of the objects of interest in the field of view. The acquired images or image sequences may be further processed in an imaging processing software provided by a computer system as part of the automated liquid handling system. Image processing may be automated such that the software automatically corrects for laser beam intensity profiles, detects and/or tracks particles in the field of view, such as biological cells, determines the size distribution of the cells, determines stain or dye intensities of the particles or of portions of the particles, in case the particles are stained before imaging, determines ratios of different stains or dyes per particle, in case the particles are stained with two or more colors, etc.

The automated liquid handling system may further comprise a motorized microscope stage, the motorized microscope stage comprising one or more mounting sections adapted to hold the microscope sample carrier for examination of the plurality of samples under a microscope. By combining the motorized stage and the mounting sections in one component, the complexity of the overall system can be reduced, albeit potentially at higher costs compared to a motorized stage holding a second mounting device that is able to hold multiple microscope sample carriers. The microscope sample carriers can be directly transferred, e.g. from the centrifuge, to the mounting sections of the motorized stage. The mounting sections can be in particular adapted to comprise means for holding or fixing the microscope sample carrier, such that the sample deposition surfaces of the microscope sample carriers are not covered by the mounting sections. As explained above, this allows for light microscopic analysis of samples deposited onto the sample deposition surfaces.

For parallel processing, however, it is currently preferred to use a motorized stage which is able to hold a second mounting device as described above.

The automated liquid handling system may further comprise means for microscopically examining the at least one sample, preferably one or more inverted microscopes. Inverted microscopes are in particular suitable for imaging samples deposited onto the microscope deposition surfaces of the microscope sample carriers, as the samples, such as biological cells, are deposited in a flat layer. Also, using inverted microscopes, there is no need of drying the sample deposition surface before imaging, as the surface is directly imaged from bottom-up such that interfering signals of the liquid (e.g. of the washing buffer) remaining in the well are avoided. Moreover, in contrast to upright microscopes, the height of the lateral walls and the supernatant within the wells of the microscope sample carrier are not limiting the imaging of the samples, in particular at higher magnification. Moreover, use of an inverted microscope setup is advantageous for the automation of sample analysis, as it allows free movement of the microscope sample carriers across the automatic liquid handling system, independent from the position of the microscope module.

The first and/or second mounting device and/or mounting sections may be adapted to hold a plurality of microscope sample carriers in parallel. Such parallel arrangement of microscope sample carriers allows for a high-throughput processing of the samples. The parallel arrangement may include holding the plurality of microscope sample carriers in one plane. This facilitates the microscopic examination of the samples, as the one or more microscopes can image the same focal plane.

The automated liquid handling system may comprise a microscope module which comprises at least a motorized microscope stage, preferably a motorized stage as defined above, and means for microscopically examining the at least one sample, preferably one or more inverted microscopes, wherein the microscope module is arranged in a fixed position within the automated liquid handling system. The position may be pre-defined to a certain, generally variable position by the user of the automated liquid handling system.

The automated transportation device may be adapted for transporting the at least one microscope sample carrier across the automated liquid handling system in x, y and z direction, and adapted for transporting the at least one microscope sample carrier to the microscope module, wherein the microscope module is physically decoupled from the automated transportation device.

Accordingly, by physically decoupling the microscope module from the automated transportation device, there is no physical contact between the microscope module and the automated transportation device. Thus, the automated transportation device does not interfere with the microscope module. This ensures that no vibration/shock interference between the automated transportation device and the microscope module occurs.

The automated liquid handling system may further comprise an incubator adapted to incubate the microscope sample carrier at a predefined temperature and/or atmosphere. The incubator may be coupled to the first and/or second mounting device and/or to the one or more mounting sections to allow incubation of samples in the microscope sample carrier at the specific temperature and atmospheric conditions. In particular, the incubator may allow incubation of the microscope sample carrier, and thus any samples therein, at a temperature selected in the range from 10° C. to 50° C., preferably 20° C. to 40° C., more preferably at about 37° C. The temperature depends on the biological sample comprised in the microscope sample carrier. The incubator may further allow incubation at a CO₂ concentration of between O and 20%, preferably between 2 and 10%, more preferably about 5%. Such concentration allows that the biological samples, in particular mammalian cell cultures, comprised in an adequate buffer are kept at a suitable pH.

Further, the storage container may be equipped with an incubator which allows incubation of the samples and/or liquids stored in the storage containers at predefined temperatures and/or atmospheres.

The centrifuge of the automated liquid handling system may be provided as described in patent application WO 2013/117606.

Specifically, the centrifuge adapted to centrifuge the microscope sample carrier may comprise a sample carrier receptacle, which can be rotated around a rotation axis R, and which has a holding section into which the microscope sample carrier can be inserted in a loading procedure, and from which the microscope sample carrier can be removed in an unloading procedure. The sample carrier receptacle may be embodied for holding one or more microscope sample carriers. In particular, the sample carrier receptacle may be embodied for holding one microscope sample carrier.

The one or more microscope sample carriers may be extending substantially parallel to the rotation axis R, i.e. the wells of each microscope sample carrier may be arranged in an axis parallel to the rotation axis R.

The centrifuge may further comprise a centrifuge platform, which is embodied for setting up the centrifuge. The centrifuge platform may be oriented parallel to the rotation axis.

The rotation axis of the centrifuge may be oriented horizontally. The horizontal axis allows the arrangement of several centrifuge modules on a platform, wherein each centrifuge can be separately controlled. Thereby, it is possible to centrifuge several microscope sample carriers individually from each other and they have not to be combined in a common batch (random access processing). The horizontal axis is rotatably fixed with both ends. Thereby, a larger degree of unbalance can be handled in comparison to a centrifuge with a horizontal rotating axis which is only fixed with one end.

The rotation axis preferably passes through the sample carrier receptacle eccentrically.

The sample carrier receptacle may be mounted to a centrifuge housing at two bearing points which are spaced apart from each other in the direction of the rotation axis R, wherein the sample carrier receptacle is able to rotate around the rotation axis R relative to the housing and wherein the holding section is provided between the bearing points.

Preferably, the rotation axis of the sample carrier receptacle coincides with the rotation axis of an output shaft of a rotary drive unit, in particular an electric rotary drive unit. In this case, the drive unit can drive the sample carrier receptacle directly, i.e. without an interposed speed-increasing or speed-decreasing transmission. This not only further reduces the number of parts required, it also produces a sample carrier centrifuge that takes up an advantageously small amount of space so that it can also be used in laboratories in which only a small amount of space for setting up laboratory devices is (still) available.

The centrifuge can be provided with a centrifuge housing equipped with an access opening that can be closed and opened by means of a cover movably mounted to the centrifuge housing. Preferably, a separate drive motor for opening and closing the access opening by means of the cover is provided, which, particularly with the above-mentioned direct coupling of the sample carrier receptacle to the output shaft of a rotary drive unit can be provided next to the rotary drive motor of the sample carrier receptacle without taking up additional space that would increase the size of the centrifuge housing. For example, the drive motor for the cover can also be an electric drive motor whose output shaft can be oriented parallel to the output shaft of the rotary drive unit for the sample carrier receptacle.

In order to process a plurality of sample carrier receptacles, which are awaiting centrifuging at different time intervals that are shorter than the duration of centrifuging required for a single test, it is possible for the centrifuge to be equipped with a plurality of sample carrier receptacles, preferably with parallel rotation axes and particularly preferably with one centrifuge housing per sample carrier receptacle. Preferably, the sample carrier receptacles can be individually driven.

Although the centrifuge modules can in fact also be basically arranged with coinciding, i.e. coaxial, rotation axes, the parallel arrangement of rotation axes is preferable because otherwise, sample carrier rotary drive units are situated between successive sample carrier receptacles, as a result of which the modularly constructed sample carrier centrifuge can be complex in appearance. In the preferred case of parallel rotation axes, the sample carrier receptacles can be placed next to one another in a very limited space, thus facilitating their automated loading and unloading so that the sample carriers to be centrifuged no longer have to be moved by operating personnel but can instead be moved by automated devices, thus advantageously reducing the risk of contamination of the samples in the sample carrier.

For the sake of facilitating an automated handling of sample carriers and a particularly desired automated loading and unloading of the modularly constructed sample carrier centrifuge, it is possible for the rotation axes of the plurality of sample carrier receptacles to be essentially situated in a common rotation axis plane. Preferably, the platform of the sample carrier centrifuge is then parallel to the rotation axis plane.

It is thus conceivable to produce a centrifuge arrangement in which the loading and unloading of one or more sample carrier receptacles can be carried out by the sample transportation device of the automated liquid handling system.

The automated liquid handling system may further comprise the at least one microscope sample carrier. Thus, the microscope sample carrier can be an integral part of the automated liquid handling system.

The plurality of sample deposition wells may be arranged such that the sample deposition surfaces are in essentially one plane. By arranging the plurality sample deposition surfaces in one plane, automated microscopic analysis of each sample deposition surface can be performed. In this case, only a minor adjustment of the focus is required as the surfaces are essentially in one plane. Accordingly, the scanning of the surfaces and image acquisition can be performed in a fast manner.

The plurality of sample deposition wells may be arranged in a regular pattern, such that the distance between neighboring sample deposition surfaces is constant. Such arrangement allows that a parallelized automated transportation device with constantly separated pipetting channels can be used for the simultaneous application of the plurality samples onto the microscope sample carrier.

The sample deposition surfaces may be plane. By using a plane sample deposition surface, an even distribution of the sample to be deposited can be achieved. This arrangement is advantageous, in case the samples to be deposited onto the sample deposition surfaces are biological cells, which should be deposited in a uniform monolayer.

Each sample deposition well may have a tapered shape towards the sample deposition surface. By forming each well in a tapered shape, the surface area of the sample deposition surface can be chosen sufficiently small for use as field-of-view during microscopic analysis. Yet, the relatively large top opening allows easy access of the wells for aspiration and/or dispensing. A small surface area of the sample deposition surface also allows the deposition of only small sample volumes or low concentrations of objects of interest for microscopic analysis, e.g. low concentrations of cells of a biological sample.

The microscope sample carrier may be partially or fully composed of an opaque material, preferably wherein the lateral walls of the microscope sample carrier are composed of an opaque plastic material. Such an opaque material, which has a low light transmittance value, such as below 10% for wavelengths typically used in light microscopy for biological samples (about 450 nm to 650 nm), prevents optical interference such as light scattering and reflection from the adjacent wells during imaging.

The sample deposition surfaces may be composed of a transparent material, in particular a transparent plastic material suitable for light and/or fluorescence microscopy. Such transparent, e.g. plastic material, typically has a light transmittance of at least 50% for wavelengths used in light microscopy for biological samples (about 450 nm to 650 nm).

The light transmittance value of the microscope deposition surfaces for wavelengths between 450 nm and 650 nm may be higher than the light transmittance value of the lateral walls of the deposition wells. As explained above, this reduces light scattering issues and ensures high quality imaging.

Each sample deposition surface may have an area of between 0.5 mm² and 20 mm², preferably between 1 mm² and 15 mm², and most preferably between 6.6 mm² and 11.18 mm². Such areas allow for one or more fields of view per surface, depending on the microscope objectives used.

Each sample deposition well may have a volume of between 2 μl and 700 μl, preferably between 5 μl and 500 μl, more preferably between 20 μl and 60 μl. These volumes are typically sufficient for processing samples, in particular cell suspensions. Depending on the concentration of particles of interest in the samples, such as specific cell types within cell suspensions, the samples can be directly applied into the microscope sample carriers for centrifugation, or alternatively, the samples can be pre-concentrated, e.g. by use of a gradient density centrifugation step.

The microscope sample carrier may be molded as a unitary body from suitable plastic materials, such as polystyrene, polyacrylate, polymethacrylate, acrylonitrile-styrene copolymers, nitrile-acrylonitrile-styrene copolymers, polyphenyleneoxide, phenoxy resins, cellulose acetate propionate, cellulose acetate butyrate and the like. The microscope sample carrier may also be prepared by an additive manufacturing method. The microscope sample carrier may also be prepared from glass. In case the microscope deposition surfaces and the lateral walls of the wells are composed of materials of different light transmittance values, the microscope sample carrier can be unitarily formed in a two-material injection process, e.g. during additive manufacturing. Alternatively, the sample deposition surfaces and the lateral walls by be formed in separate processes and bonded by means of ultrasonic welding, gluing, etc. Each sample deposition well may be defined by an angle formed between the one or more lateral walls and the sample deposition surface, wherein the angle is between 70° and 110°, preferably between 80° and 100°, most preferably about 90°. Generally, it is preferred that the angle between the lateral walls and the sample deposition surfaces is as close to 90 degrees as possible. The angle typically depends on the manufacturing technique, e.g. molding technique, which is applied.

Each sample deposition surface may have an area of between 0.5 mm² and 20 mm², preferably between 1 mm² and 15 mm², and more preferably between 6.6 mm² and 11.18 mm². Each sample deposition surface may have a thickness between 0.1 and 0.4 mm, preferably between 0.15 and 0.35 mm, more preferably of about 0.3 mm.

Alternatively, the thickness may be about 0.13 to 0.17 mm, or about 0.17 to 0.19 mm or about 0.17 to 0.25 mm, which are comparable to conventional coverslip thicknesses (e.g. coverslips #1.5 or #2), in particular the thickness may be about 0.17 to 0.25 mm. Such thinner thicknesses are, however, more expensive to produce compared to thicknesses of about 0.3 mm.

Preferably, the standard deviation in thickness may be less than 0.08 mm, preferably less than 0.05 mm, more preferably less than 0.01 mm.

The sample deposition surfaces may be arranged in one or more rows. Thereby, common automated pipetting systems with pipetting tips arranged in a row may be used for the deposition of the samples onto the sample deposition surfaces.

The automated transportation device may comprise a robotic arm or robotic gripper for receiving one or more flanges or recesses of the coupling section of the microscope sample carrier. The microscope sample carrier can, thus, be easily transferred across the system.

The present invention also relates to a method for processing a plurality of samples in at least one microscope sample carrier, wherein the microscope sample carrier comprises a plurality of sample deposition wells, wherein each sample deposition well is defined on its lateral sides by one or more lateral walls and on its bottom side by a sample deposition surface, the method carried out by an automated liquid handling system, the method comprising:
applying, by an automated transportation device of the automated liquid handling system, each biological sample of a plurality of biological samples into at least one sample deposition well of the plurality of sample deposition wells;
separating, by a centrifuge of the automated liquid handling system, a plurality of first portions from a plurality of second portions of the plurality of the biological samples by means of application of a centrifugal force, wherein the plurality of first portions is deposited on the plurality of sample deposition surfaces;
transporting, by the automated transportation device of the automated liquid handling system, the microscope sample carrier across the automated liquid handling system, wherein the automated transportation device is configured to couple with a coupling section of the microscope sample carrier.

The method according to the present invention allows the application of multiple samples, e.g. of the same or different origin, onto one microscope sample carrier. The samples may be in particular liquid samples, and preferably comprising biological cells in suspension. The combination of sample deposition and separation of portions enables an overall high-throughput process of sample preparation for microscopic analysis. The separation step in particular allows for the proper deposition of samples onto the sample deposition surface. In the method, the sample deposition surfaces are adapted to hold a plurality of first portions of samples, such as the biological cells of a cell suspension. The first portions of a sample being deposited on the sample deposition surfaces can be analyzed microscopically, for example using a light or fluorescence microscope. Furthermore, after the separating step, the plurality of second portions, e.g. liquids, such as buffers, stains, or wash solutions may be efficiently removed, if needed, e.g. by subsequent aspiration. In general, however, a step of removing the second portions from the wells is optional, in particular in case an inverted microscope setup is used for the examination of the first portions of the samples, where only the sample deposition surfaces are imaged and interfering signals from above are efficiently excluded in the optical path of the microscopes.

In the step of separating, the plurality of surfaces may be in a position perpendicular to the axis of rotation. Thereby, the first portions can be radially and evenly spread onto the sample deposition surfaces.

The plurality of first portions may be deposited onto the plurality of sample deposition surfaces in uniform layers. In particular, the first portions may comprise cells, which may be deposited in uniform layers of single-cell thickness. This allows accurate imaging of the samples.

One or more or all of the sample deposition surfaces and/or the inner surfaces of the lateral walls of the microscope sample carrier may be prepared to specifically react with the plurality of first portions or second portions of the biological samples. Thereby, the first portions or the second portions are not only separated by means of centrifugation, but also by means of reactive surfaces which either specifically react with the first or second portions. Also, as the sample deposition surfaces form the bottoms of the wells, the user can coat the surfaces with reagents or proteins, such as antibodies, that react with the sample. Thereby, the wells can be used as incubation chambers, and reaction of the reagents or proteins with parts of the samples can be read, for example using colorimetric assays.

The sample deposition surfaces may be in particular coated with adhesion promoters that increase the adhesion of biological cells to the surface. Adhesion promoters may provide in particular hydrophilic surfaces, such as gelatin, aminoalkylsilane or poly-L-lysine. The sample deposition surfaces and/or the inner surfaces of the lateral walls of the microscope sample carrier may be alternatively or in addition coated with antibodies that react with the first portion and/or second portion of the sample.

The method may further comprise at least one of the following steps:
fixing the deposited plurality of first portions;
staining the deposited, preferably fixed plurality of first portions;
washing the deposited, preferably fixed plurality of first portions;
drying the optionally stained or washed deposited plurality of first portions by removal of supernatants,
incubating, by means of an incubator, the plurality of samples and/or plurality of first portions at a predefined temperature and/or atmosphere for a predefined time interval.

The step of drying may comprise centrifuging the microscope sample carrier, preferably at a centrifugal force of 50 to 500 g and/or for a centrifugation time of between 0.5 and 5 min.

The incubator may allow incubation of the microscope sample carrier, and thus any samples therein, at a temperature selected in the range from 10° C. to 50° C., preferably 20° C. to 40° C., more preferably at about 37° C. The temperature depends on the biological sample comprised in the microscope sample carrier. The incubator may further allow incubation at a CO₂ concentration of between o and 20%, preferably between 2 and 10%, more preferably about 5%. Such concentration allows that the biological samples, in particular mammalian cell cultures, comprised in an adequate buffer are kept at a suitable pH.

Preferably, the step of drying comprises aspirating supernatants from the microscope sample wells. Aspirating, e.g. by means of the automated transportation device, ensures that the first portions remain properly deposited onto the sample deposition surfaces, also in case the first portions are not strongly adhered to the sample deposition surfaces. This is in particular important for sensitive biological cells, which do not adhere to the sample deposition surfaces by means of an adhesion promoter.

The method may further comprise at least one of the following steps:
transporting, by the automated transportation device of the automated liquid handling system, the microscope sample carrier across the automated liquid handling system to a mounting device adapted to hold the at least one microscope sample carrier for examination of the plurality of biological samples and/or first portions under a microscope, preferably to a second mounting device as defined above, more preferably wherein the mounting device is held and/or its position adjusted by a motorized microscope stage as defined above;
transporting, by the automated transportation device of the automated liquid handling system, the microscope sample carrier across the automated liquid handling system to a motorized microscope stage, the motorized microscope stage comprising one or more mounting sections adapted to hold the at least one microscope sample carrier for examination of the plurality of samples under a microscope;
microscopically analyzing the plurality of biological samples and/or plurality of first portions, preferably by means of one or more inverted microscopes, preferably by means of a microscope module as defined above.

The method, thus, may include the automated microscopic analysis of the plurality of samples and/or first portions. Accordingly, the automated transportation device can transport and position the samples within the microscope sample carrier onto a mounting device, such as the second mounting device as defined above, and the motorized microscope stage is arranged to hold and adjust the mounting device within the optical path of one or more microscopes, in particular of one or more inverted microscopes. The motorized microscope stage may allow scanning of each sample deposition surface along multiple field of views by controlled movement in x and y direction.

Microscopic analysis may include automated image analysis of the plurality of samples and/or first portions.

The method may be performed by the automated liquid handling system as described above.

The present invention also relates to a method for culturing biological cells in at least one microscope sample carrier, wherein the microscope sample carrier comprises a plurality of sample deposition wells, wherein each sample deposition well is defined on its lateral sides by one or more lateral walls and on its bottom side by a sample deposition surface, the method carried out by an automated liquid handling system, the method comprising:
applying, by an automated transportation device of the automated liquid handling system, each biological sample of a plurality of biological samples into at least one sample deposition well of the plurality of sample deposition wells;
incubating, by an incubator of the automated liquid handling system, the plurality of biological samples.

The method according to the present invention allows the processing and culturing of biological cells in an automated manner. The cells can thus be incubated directly in the microscope sample carriers, and analyzed in down-stream applications, such as in microscopic assays.

The biological samples may be obtained by an upstream processing method carried out by the automated liquid handling system. The method may further comprise one or more of the steps:
separating, by a centrifuge of the automated liquid handling system, a first fraction of biological cells in a first centrifugation tube;
aspirating, by an automated transportation device, the first fraction of the biological cells from the first centrifugation tube;
transferring, by the automated transportation device of the automated liquid handling system, the first fraction of the biological cells to a second centrifugation tube;
suspending the first fraction of biological cells in the second centrifugation tube in a suitable buffer;
separating, by a centrifuge of the automated liquid handling system, a second fraction of biological cells in the second centrifugation tube;
aspirating, by an automated transportation device, the second fraction of the biological cells from the second centrifugation tube;
transferring, by the automated transportation device of the automated liquid handling system, the second fraction of the biological cells to a microscope sample carrier.

The second fraction of biological cells may be processed by incubation, as described above.

The method may further comprise one ore more of the steps:
separating, by a centrifuge of the automated liquid handling system, a plurality of first portions from a plurality of second portions of the plurality of the biological samples by means of application of a centrifugal force, wherein the plurality of first portions is deposited on the plurality of sample deposition surfaces;
fixing the deposited plurality of first portions;
staining the deposited, preferably fixed plurality of first portions;
washing the deposited, preferably fixed plurality of first portions;
drying the optionally stained or washed deposited plurality of first portions by removal of supernatants.

The method for culturing biological cells may be combined with the method for processing a plurality of samples in at least one microscope sample carrier, as described above.

The present invention further relates to a use of the method as described above for the isolation and microscopic examination of biological samples.

The biological sample can be any fluid, gel or solution containing biological elements. For instance, the biological sample from which rare cells are to be extracted can be any body fluids from a human or animal or a dispersion of a cellular tissue. Examples thereof are blood, in particular peripheral blood such as venous or arterial blood, lymph, urine, exudates, transudates, spinal fluid, seminal fluid, saliva, fluids from natural or unnatural body cavities, bone marrow and dispersed body tissue. The most preferred body fluid is peripheral blood. The biological samples may comprise cells, blood cells, cord blood cells, bone marrow cells, erythrocytes, leukocytes, lymphocytes, epithelial cells, stem cells, cancer cells, tumor cells, circulating tumor cells, cell precursors, hematopoietic stem cells, mesenchymal cells, stromal cells, platelets, sperms, eggs, oocytes, microbes, microorganisms, bacteria, fungi, yeasts, protozoans, viruses, organelles, nuclei, nucleic acids, mitochondria, micelles, lipids, proteins, protein complexes, cell debris, parasites, fat droplets, multi-cellular organisms, spores, algae, clusters or aggregates of the above, which may be microscopically analyzed.

### 4. BRIEF DESCRIPTION OF THE FIGURES

Aspects of the present invention will be explained in more detail with reference to the accompanying figures in the following. These figures show:

FIG. 1a-d: schematic representations of a microscope sample carrier according to an embodiment of the present invention;

FIG. 2a, b: schematic representations of an automated liquid handling system according an embodiment of to the present invention;

FIG. 3a-d: schematic representations of a part of a centrifuge according to an embodiment of the present invention;

FIG. 4a-j: schematic representations of parts of an automated liquid handling system according to an embodiment of the present invention;

FIG. 5: scanning mode for the sample deposition surface of a microscope sample carrier according to an embodiment of the present invention;

FIG. 6: workflow of a method according to an embodiment of the present invention;

FIG. 7a-f: schematic representations of parts of an automated liquid handling system according to an embodiment of the present invention.

### 5. DETAILED DESCRIPTION OF CURRENTLY PREFERRED EMBODIMENTS

In the following, embodiments and variations according to the present invention are described in more detail. It is, however, emphasized that the present invention is not limited to these embodiments and variations. It is also mentioned that in the following only individual embodiments of the invention can be described in more detail. The skilled person will realize, however, that the features described in relation to these specific embodiments of the microscope sample carrier, the sample capture rack or the method may also be modified or combined in a different manner within the scope of the invention, and that individual features may also be omitted if these seem dispensable in a given case.

The present invention relates to an automated liquid handling system for processing a plurality of samples in at least one microscope sample carrier, as well as a method for such processing, which is carried out by an automated liquid handling system.

In particular, the present invention allows the preparation of thin-layer smears of single cell thickness of biological fluids for diagnostic evaluation in high-throughput. Thereby, a high quality, undistorted cell smear can be created on a microscope slide surface, having a high numerical density of cells available for differential counting and morphological, histochemical, fluorescent, autoradiographic and various other types of biological tests. Moreover, as the sample deposition surfaces are arranged as bottoms of wells, these wells of the microscope sample carrier can be used as reaction containers or vessels, e.g. for culturing microorganisms and cells.

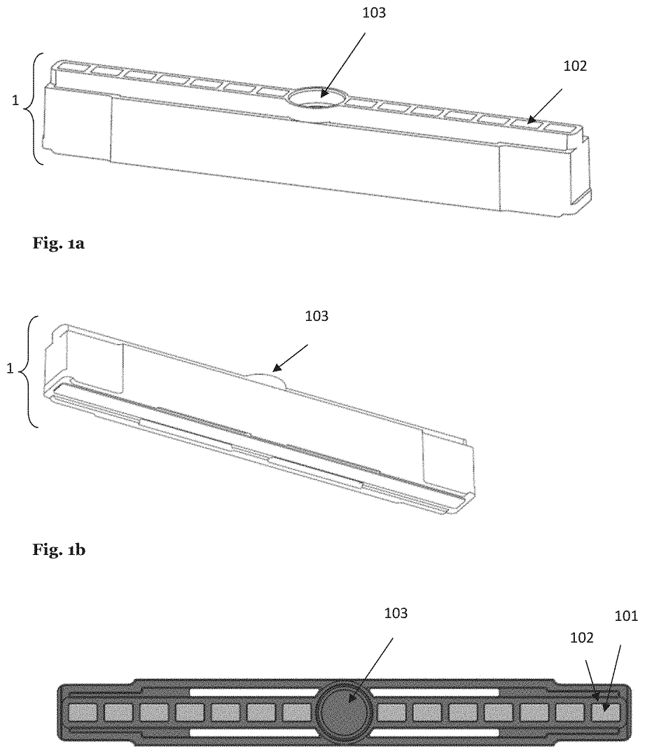

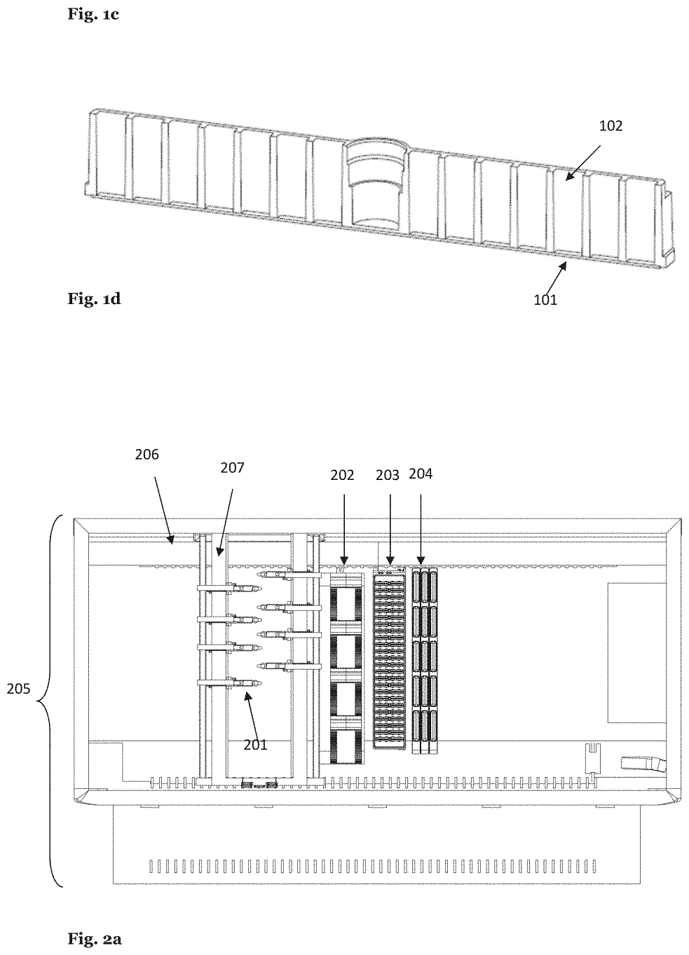

FIG. 1a shows a schematic representation of an embodiment of the microscope sample carrier (1) according to the present invention. In this embodiment, 14 sample deposition surfaces (101, not shown) are arranged regularly, i.e. with equal distance between neighboring sample deposition surfaces, in one plane and furthermore in one row. It is, however, also conceivable that the sample deposition surfaces are arranged in multiple rows, e.g. analogous to a 96-well system. Further, in this embodiment, the sample deposition surfaces are provided as flat surfaces, and each sample deposition surface forms the bottom surface of a well (102). Thus, each deposition surface is physically separated from adjacent deposition surfaces. The microscope sample carrier (i) according to FIG. 1a comprises a coupling section (103), which is arranged in the center of the row formed by the plurality of wells (102), wherein the coupling section (103) may be compatible with automated pipetting channels, such as CO-RE, to enable handling of the microscope sample carrier without interfering with the sample deposition surfaces. However, also other ways of handling the microscope sample carrier by robotic arms or grippers are conceivable.

FIG. 1b shows a side view of the microscope sample carrier of FIG. 1a.

FIG. 1c shows a top view of the microscope sample carrier (i) of FIG. 1a.

FIG. 1d shows a cross-sectional view of the microscope sample carrier (i) of FIG. 1a. As illustrated in FIG. 1d, the wells (102) can have a straight shape from the top towards the bottom surfaces forming the sample deposition surfaces. Alternatively, it is also conceivable that the wells (102) have a tapered shape towards the bottom surfaces.

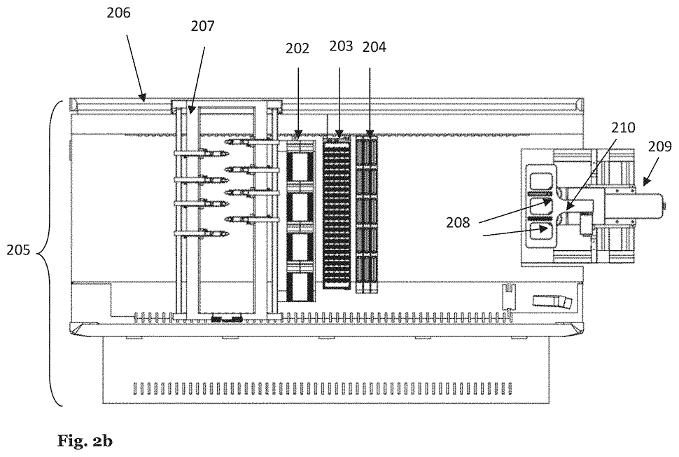

FIG. 2a shows a top view of a schematic representation of an embodiment of the automated liquid handling system according to the present invention. In this embodiment, the automated liquid handling system comprises a pipetting channel module (201), a centrifuge module (202), a first mounting device adapted to hold a plurality of microscope sample carriers (203) and storage containers for receiving and/or storing the one or more samples and/or one or more liquids (204). The pipetting channel module (201) comprises an automated transportation device to transfer a plurality of samples or liquids into and out of the microscope sample carriers. Moreover, the pipetting channel module (201) is arranged to comprise an automated transportation device, which allows transport of the microscope sample carriers across the system. The different components are positioned onto a platform (205) comprising a transfer section with guide rails (206) in the x direction and guide rails (207) in the y direction. The pipetting channels of the pipetting channel module (201) are attached to the transfer section, such that movement in x and y axis is possible. Further, the pipetting channels can be moved in vertical direction to pick up and transport the microscope sample carriers across the platform and/or to pick up liquids or samples from the storage containers.

FIG. 2b shows a top view of a further schematic representation of an embodiment of the automated liquid handling system according to the present invention. According to this embodiment, the automated liquid handling system comprises, in addition to the elements mentioned above with reference to FIG. 2a, two inverted microscopes (208) and a motorized microscope stage (209) which is movable in the xy-plane. The motorized stage comprises a second mounting device (210) for placing and holding two microscope sample carriers. It is also conceivable that the second mounting device may hold one microscope sample carrier, or that the second mounting device is adapted to hold three or more microscope sample carriers, thereby facilitating parallel viewing and microscopic examination of a plurality of samples deposited in a plurality of microscope sample carriers. Alternatively, each microscope may comprise a motorized stage and a suitable second mounting device for the microscope sample carrier, or a motorized stage comprising a mounting section.

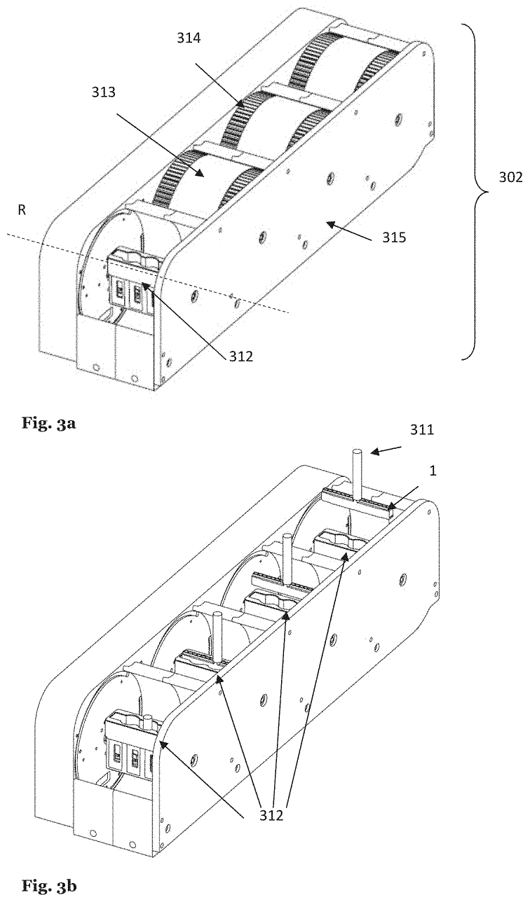

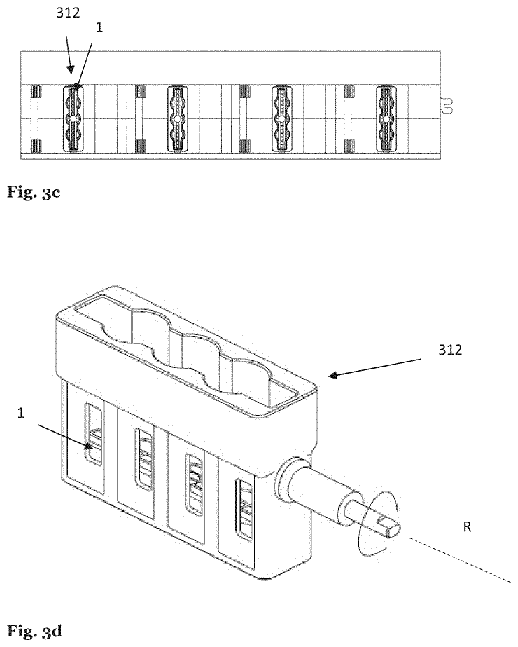

FIG. 3a-d show schematic representations of a centrifuge (302) adapted to centrifuge the microscope sample carrier.

As shown in FIG. 3a, the centrifuge according to this embodiment comprises four sample carrier receptacles (312) arranged in parallel rotation axes. Each sample carrier receptacles (312) is connected to a rotation drive unit (not shown) which performs rotation of the sample carrier receptacles (312) around the rotation axis R during operation. Further, during operation, each sample carrier receptacle (314) may be covered by a cover (313) movably mounted to a centrifuge housing which encloses the centrifuge. Each sample carrier receptacle is adapted to hold one microscope sample carrier. It is, however, also conceivable that less or more than four sample carrier receptacles are comprised in the centrifuge, and/or that a sample carrier receptacle can hold two or more microscope sample carriers.

During operation, the cover protects the samples in the microscope sample carriers loaded into the sample carrier receptacles. Preferably, a separate drive motor for opening and closing the cover is provided. The cover (313), preferably on its large circumference surface, can have at least one engagement formation (314), preferably a plurality of engagement formations (314), for example in the form of a denticulation, that a counterpart engagement formation, e.g. a gear, provided in the centrifuge housing can drive with form-locked engagement to execute an opening and closing motion in order to enable an opening or closing of the cover.

In FIG. 3b, the centrifuge is shown during loading of four microscope sample carriers (1) into four sample carrier receptacles (312). In this embodiment, the automated transportation device comprises pipetting channels (311) which transfer the microscope sample carriers (1) into each one sample carrier receptacle, such that the microscope sample carriers are fully integrated into the receptacles during centrifugation (as shown for the front microscope sample carrier). After transfer, the pipetting channels (311) disconnect from the microscope sample carriers, such that the centrifugation step can be started.

FIG. 3c shows a schematic top view of the centrifuge module (302) with four microscope sample carriers loaded into four sample carrier receptacles.

FIG. 3d shows a further embodiment of a sample carrier receptacle, loaded with a microscope sample carrier. When positioned into the centrifuge, e.g. the centrifuge as shown in FIG. 3a to c, the sample carrier receptacle is rotated along the rotation axis R, as indicated.

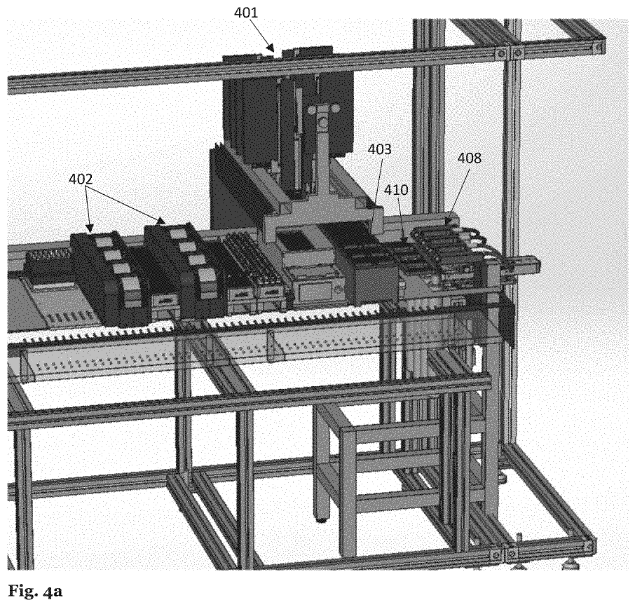

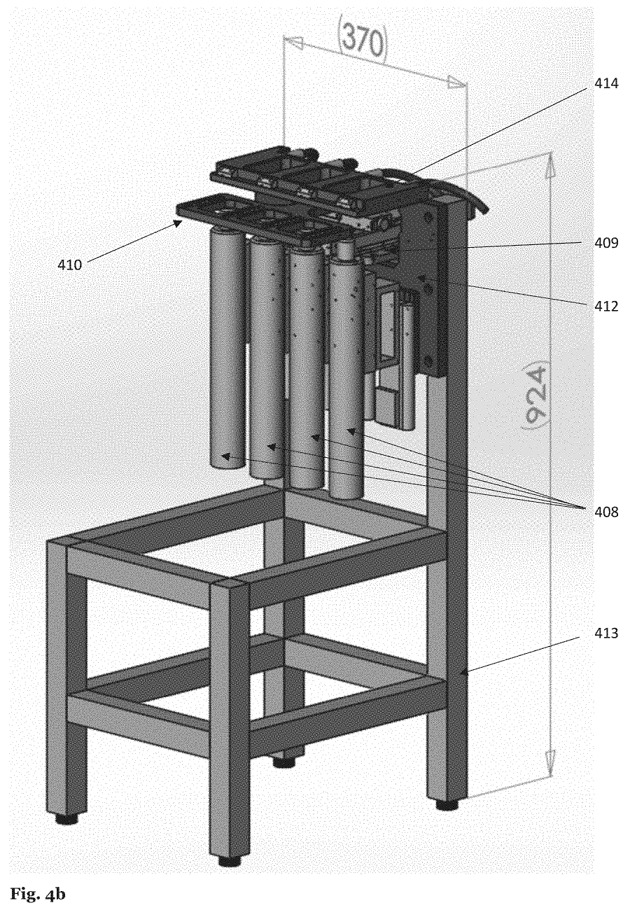

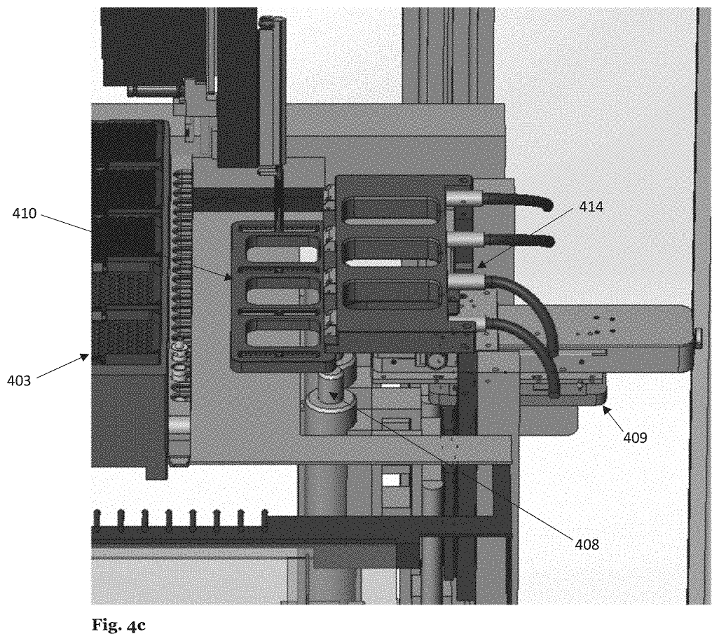

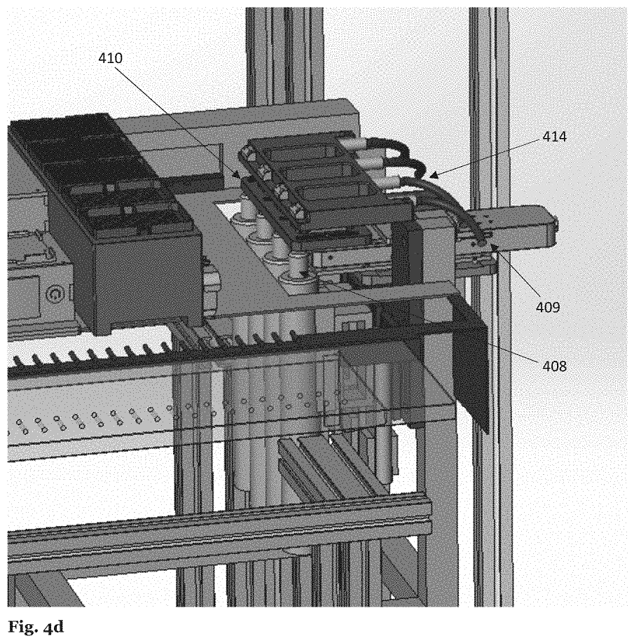

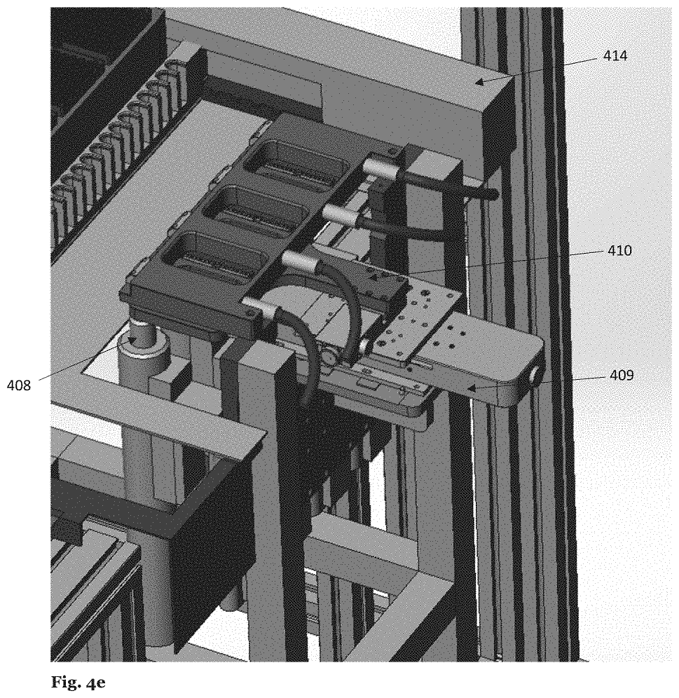

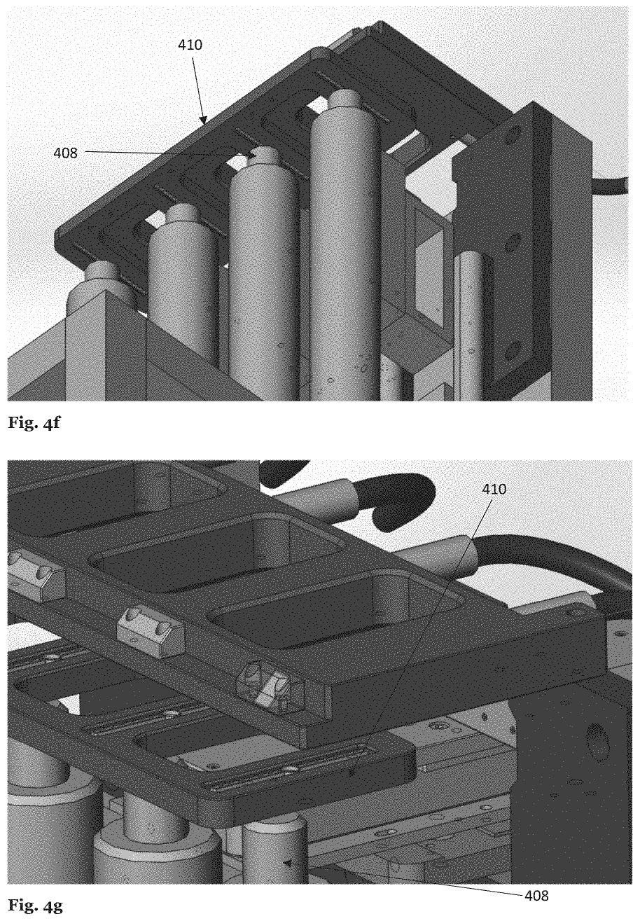

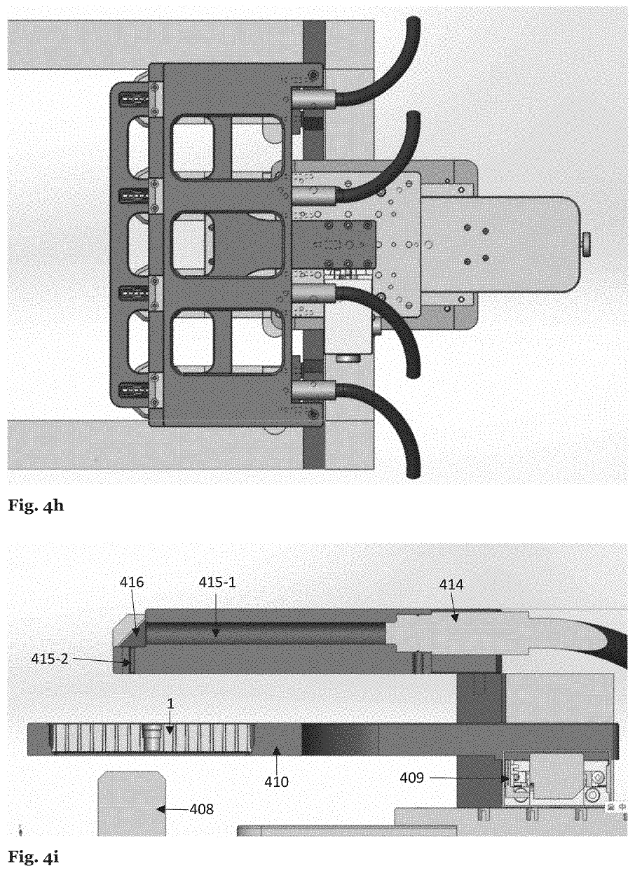

FIG. 4a shows a further view of an automated liquid handling system according an embodiment of the present invention. According to this embodiment, the automated liquid handling system comprises a pipetting channel module (401), two centrifuge modules (402), a first mounting device for holding a plurality of microscope sample carriers (403) and storage containers for receiving and/or storing the one or more samples and/or one or more liquids (not shown). The pipetting channels are attached to the transfer section, such that movement in x and y axis is possible. As described above, the pipetting channels can be moved in vertical direction to pick up and transport the microscope sample carriers across the platform. Moreover, the automated liquid handling system comprises a microscope module with four inverted microscopes (408). Alternatively, less, e.g. one or two inverted microscopes, or more microscopes can be included in the automated liquid handling system. A second mounting device (410) allows simultaneous positioning of the microscope sample carriers for imaging. In this embodiment, the second mounting device (410) carries four microscope sample carriers in accordance with the number of microscopes.

FIG. 4b shows a schematic view of the microscope module of FIG. 4a. Four inverted microscopes (408) are attached to a wall structure (412), which is integrated into a frame structure (413). The second mounting device (410) is mounted on a motorized stage (409), which allows precise movement of the adapter in the xy-plane. Light sources (414) are provided to enable examination of the content contained in the microscope sample carriers by the one or more microscopes. In the shown embodiment, each of the four inverted microscopes (408) may have its own lights source (414). More particularly, the light source may be positioned, when viewed from the direction of centrifuge modules (402), behind the microscopes. As will be explained in further detail below, providing the light sources behind the microscopes is possible since the light beam of the light source may enter a tunnel of manifold means in an essentially horizontal direction (x direction), wherein a light reflecting object (such as prism) projects the light beam in a vertical direction (y direction) downwards to the microscope sample carriers, or, more particularly, to the sample deposition surfaces forming the bottom surfaces of the well(s) of the microscope sample carrier(s). Although the microscope module is integrated with the automated liquid handling system, it is not in the sense that the entire microscope module has any physically contact with the automated liquid handling system. This ensures that no vibration/shock interference by the centrifuge or movement of other components occurs. The relative positioning of the microscope module and the liquid handling system can be pre-defined and fixed to ensure robotic precision during operation.

FIG. 4c and FIG. 4d show schematic views of the microscope module, integrated into the automated liquid handling system, before (FIG. 4c) and after (FIG. 4d) the microscope sample carriers are transferred into the field of view of the microscope objectives. In FIG. 4c, four microscope sample carriers are positioned into the second mounting device (410) by means of an automated transportation device. Specifically, as illustrated in FIG. 4c, one pipetting channel is shown to place one microscope sample carrier into the second mounting device, while three further microscope sample carriers are already loaded. After positioning, the motorized stage allows that the second mounting device is moved into the direction of the microscopes, such that each of the four microscope sample carriers is positioned onto the respective objective of the microscopes. According to the exemplary view of FIG. 4d, the wells at the extreme left of each microscope sample carrier are positioned on top of each microscope unit.

FIG. 4e shows the microscope module of FIG. 4d from a further top-view perspective.

FIG. 4f depicts the microscope module of the liquid handling system from a bottom-up view. In this schematic, the second mounting device (410) positions four microscope sample carriers on top of four inverted microscopes, each imaging one of the central sample deposition surfaces of the microscope sample carrier. In this embodiment, focusing of the lenses of the microscopes is realized by means of a motorized Z-axis of each inverted microscope. For example, the microscope units can comprise means for automated focusing in the Z-axis. It is, however, also conceivable that the motorized stage carrying the second mounting device for the microscope sample carriers can be moved in the z-axis for focusing.

FIG. 4g depicts the microscope module of the liquid handling system according to FIG. 4f from a more detailed lateral view. In particular, in this schematic, more details of a possible structure for the manifold means can be seen. In this embodiment, the manifold means is provided as a frame structure, providing tunnel(s) for the light beams, wherein the frame structure is provided in a plane essentially parallel to the second mounting device (410). Light reflecting means are provided at the end of each first horizontal tunnel to project a horizontally-incoming light beam in a downward vertical direction. The figure shows, as an example, a prism at the end of each of the four tunnels, which may be covered by a protective cover. The light reflecting means may offer a convenient way of focusing the light to a desired shape or diameter, depending on the shape and size of the area which should be exposed (e.g., a single sample deposition surface forming the bottom surface of the well(s) of the microscope sample carrier).

Moreover, FIG. 4g also shows that the light source is not connected from above to the manifold, rather on the back, to enable the essentially horizontal entrance of the light beam (as explained previously). Experiments have shown that this configuration provides additional advantages over other embodiments. In particular, if the light source enters the manifold from the back, there are no physical elements on top of the manifold means that may interfere with any movement of the robotic arm(s) or gripper(s). Thus, loading and unloading of the microscope sample carriers into or out of the second mounting device may be facilitated, in particular as a high degree of precision is needed for the movement of the robotic arm(s) or gripper(s). In addition, it has also been shown that the light beam(s) in this arrangement produce less heat than in other configurations.

FIG. 4h is a further top view of the illustrated embodiment, wherein each of the four inverted microscopes images one of the central sample deposition surfaces of the microscope sample carrier (as in FIGS. 4f and 4g, for example). In this illustration, the advantage described above, namely the free upper area on the top of the manifold means becomes clearly visible.

FIGS. 4i and 4j show two cross-sections of the manifold means, the microscope (408) and the second mounting device (410) of the automated liquid handling system according to the embodiments described previously. In these illustrations, the horizontally arranged first tunnel (415-1) as provided within the manifold means can be seen. During operation, the light source (414) may enter the first tunnel (415-1) on one end, to enable the light beam to essentially pass through the tunnel in a horizontal manner. At the other end of the first tunnel (415-1), the light reflecting means (416) may be provided, e.g. a prism. In vertical downwards direction, a vertically arranged second tunnel (415-2) follows after the prism (416). Thus, during operation, a light beam of the light source (414) entering the first tunnel (415-1) is reflected by the prism downwards through the second tunnel (415-2) towards the area of the microscope sample carrier that should be projected. The structure as described may be provided correspondingly for each of the number of microscope sample carriers that are processed in parallel with the second mounting device.

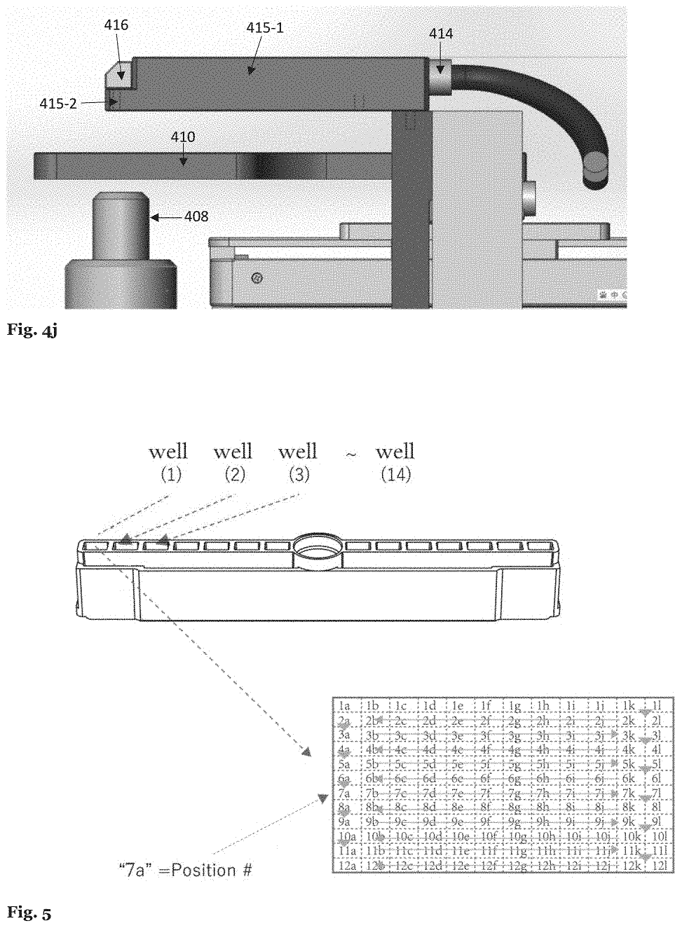

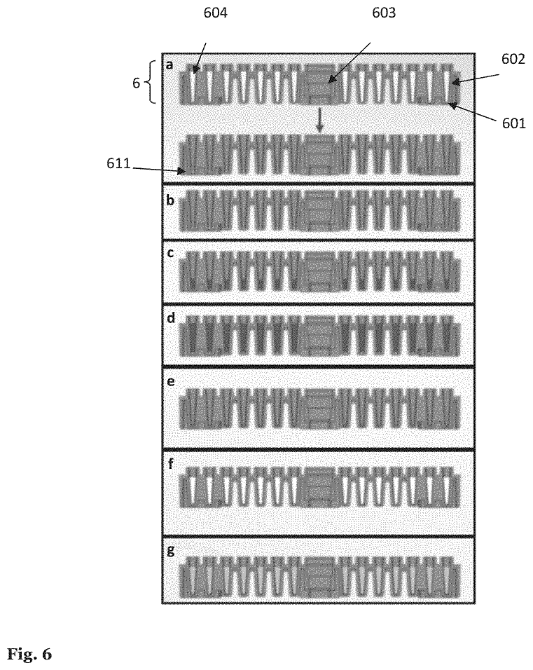

FIG. 5 illustrates an exemplary scanning of a well of a microscope sample carrier according to an embodiment of the present invention. According to this embodiment, the sample deposition surface of well No. 1 is sequentially scanned according to the scheme, namely in 12×12 field of views, following an “S” pattern. Scanning is achieved by a corresponding x-y movement of the microscope stage. However, other patterns are also conceivable and programmable. Thus, according to this embodiment, a total of 144 images are generated per well under 1000× magnification. When the second mounting device or the mounting section of the motorized stage is loaded with more than one microscope sample carrier, parallel processing enables images generation of the well in the same position on all microscope sample carriers simultaneously.

In the following, a method according to the present invention is described with reference to FIG. 6. In this method, a microscope sample carrier (1) according to an embodiment of the invention is used, which comprises a plurality of sample deposition surfaces (601), each forming the bottom surface of a well (602). At the center of the microscope sample carrier (6), a coupling section (603) compatible with automated liquid handling instruments is arranged for handling of the microscope sample carrier.

The microscope sample carrier can be, for example, constructed as explained above with reference to FIG. 1, but also other designs of microscope sample carrier according to the invention can be used.

In step a of FIG. 6, biological cell samples in liquid suspension (604) of the same source or of different sample sources are applied into the wells of the microscope sample carrier. For example, a cell suspension of about 500 μl, comprising enriched cell fractions of a blood sample, can be used for application.

After sample application, the microscope sample carrier (6) can be centrifuged at a suitable centrifugal force and time, for example, when handling blood cells, 200 g for 5 minutes, preferably with the sample deposition surfaces normal to a vertical axis of centrifuge rotation.

It is important to note the initial centrifugal acceleration speed plays a role in making sure that acceleration may follow a linear or non-linear velocity, and most importantly, it should be a gradual increase until the targeted centrifugation speed is reached, instead of a sudden motion.

The Applicant became aware of this problem when tested with default speed setting that reaches 200 g in merely one second. Due to the sudden acceleration, the sample cells first experience a drift towards the edge of the well, and once deposited at the bottom surface, instead of an even distribution at the bottom of the well, the cells are sidelined.

In the course of the tests, the Applicant then slowed down the acceleration to about 30 seconds or longer, a gradual and steady acceleration, then the uniform distribution of cells is not affected by the acceleration.

Thereby, a first portion of the sample (611), i.e. cells and microemboli within the sample, is sedimented and deposited onto the sample deposition surfaces in a uniform layer, while a second portion of the sample (not shown), in particular liquid, remains in the wells as supernatant. The centrifugation step thereby results in an increase in concentration of the cells on the sample deposition surface. After centrifugation, the supernatant may be removed from the wells by means of an automated transportation device, aspirating the supernatant.

In (optional) step b, the microscope sample carrier is dried, e.g. by means of a centrifuge. The centrifuge speed and time can be optimized accordingly for best performance. For example, the microscope sample carrier can be centrifuged at 150 g for 2 minutes. This step b is in particular optional, in case the supernatant is already removed from the wells, as described above.

In step c, the cells of the first portion of the sample are fixated. Fixatives that can be used include chemicals used for protecting biological samples from decay, and such fixatives can impede biochemical reactions occurring in the specimen and increase the mechanical strength and stability of the specimen. Various fixatives can be used including, but not limited to, methanol, ethanol, isopropanol, acetone, formaldehyde, glutaraldehyde, EDTA, surfactants, metal salts, metal ions, urea, and amino compounds. For example, 2 to 5 μl methanol can be applied to each sample deposition surface and incubated for 20-30 seconds.

In step d, the sample are stained. Staining a specimen increases the contrast of the specimen when it is viewed or imaged under a microscope or other imaging device. Romanowsky stains, Wright-Giemsa stains, Geimsa stains and/or other dyes or stains can be used, including hematoxylin and eosin, fluorescein, thiazin stains using antibodies, nucleic acid probes, and/or metal salts and ions. For example, 10 μl of staining solution can be added to each sample deposition surface and incubated for 3 minutes.

Importantly, since the sample deposition surfaces are physically independent from each other, different fixatives and staining can be applied to treat each sample without risk of cross contamination. This can be done simultaneously via multiple pipetting channels on automated liquid handling system.

For example, one identical sample can be applied to all twelve sample deposition surfaces for different downstream applications. Alternatively, twelve different samples, e.g. from different patients, can be applied to the microscope sample carrier.

In step e, the staining solution is removed from the wells, for example by aspirating by means of an automated transportation device.

In step f, the stained cells of the first portion of the sample are rinsed. Rinsing solutions include, for example, distilled water, buffered, aqueous solutions, organic solvents, and mixtures of aqueous and organic solvents, with or without buffering. For example, 10 to 20 μl of ultrapure water can be applied to each sample deposition surface and incubated for 1 min.

After washing, the supernatant may be aspirated, and the sample may be dried, e.g. by centrifugation. The parameters for centrifugation can be optimized with respect to the sample. For example, the microscope sample carrier can be centrifuged at 150 g for 2 minutes to dry the sample. In general, it is desirable to have a fast cell smear drying speed which can improve the uniformity of the cell morphology across the smear due to the fact that all cells experience the same osmolarity change during drying. Quickly drying the thin-layer allows the removal of the solvent from the thin-layer faster than the cells can react to the loss of solvent. However, aspiration and drying after washing are optional steps, in particular in case inverted microscopes are used for imaging the deposited samples, as the washing solution does not interfere with the imaging.

In step g, the microscope sample carrier is ready for imaging or any further downstream process, e.g. fluorescence in situ hybridization. Since the boundaries of cell sample and the relative spatial position (x, y, z axis) of each sample deposition surface is substantially fixed, it is convenient to program the imaging workflow for a digital microscope with motorized stage in order to capture the entire sample area and to achieve high throughput.

The method as well as the automated liquid handling system according to the present invention can make use or comprise a system comprising a microscope, a camera for acquiring microscope images, a computer system with an image processing software, centrifuge, ID-reader, pipetting channels, sample reservoirs, reservoirs for fixatives, stains, rinsing solution and a control system. In general, the system and the method disclosed herein provide for efficient, contamination-free and highly uniform specimen processing with minimized usage of fluid quantities. The method according to the invention may include one or more fixing, staining, and rinsing phases. The system can be implemented as a standalone device or as a component in a larger system for preparing and examining biological specimens. For many applications, both high throughput operation and low fluid consumption are desirable. By maintaining high throughput, specimens can be efficiently processed for subsequent examination. By keeping fluid consumption low, the amount of processing waste is reduced along with the required volume of processing reagents, keeping operating costs low.

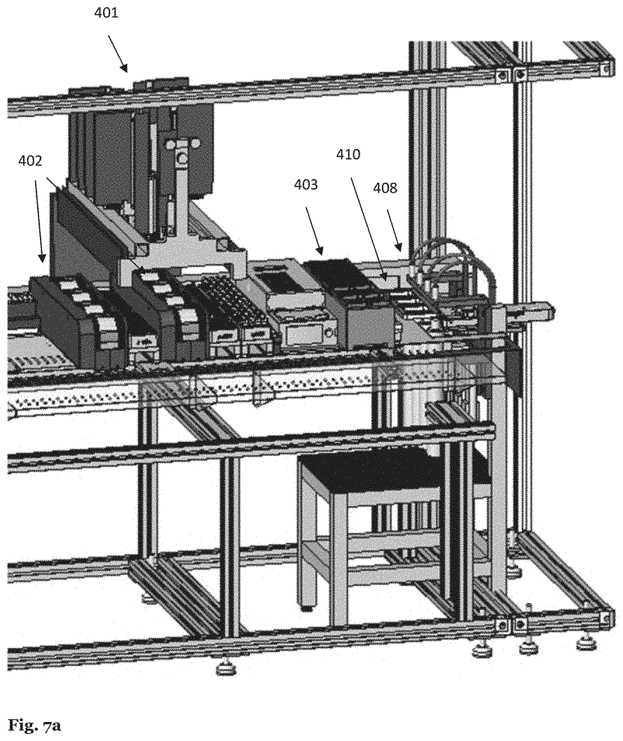

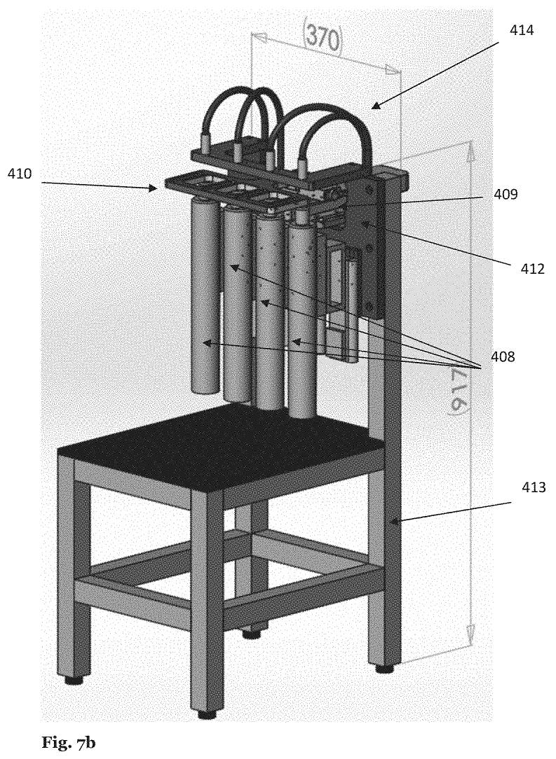

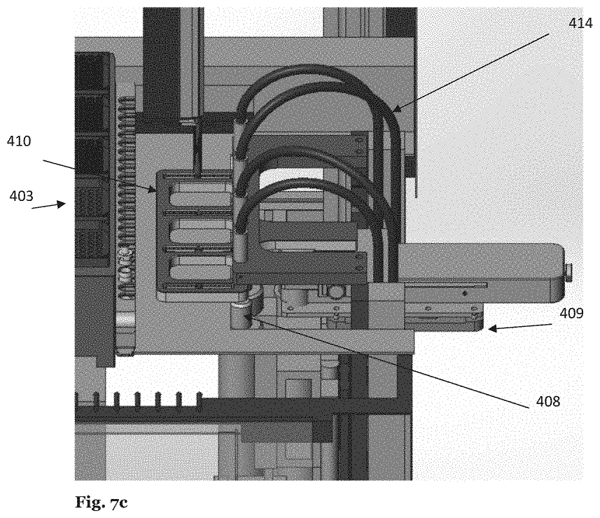

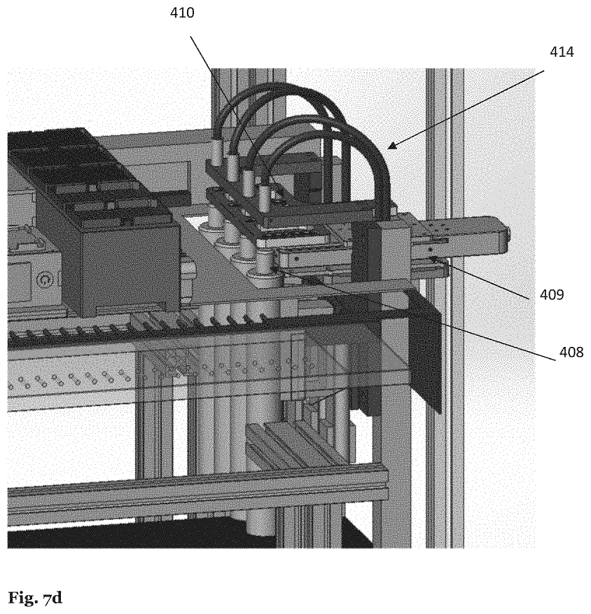

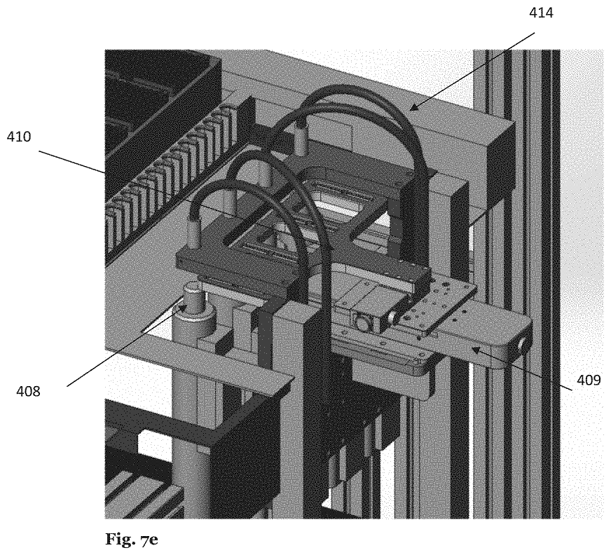

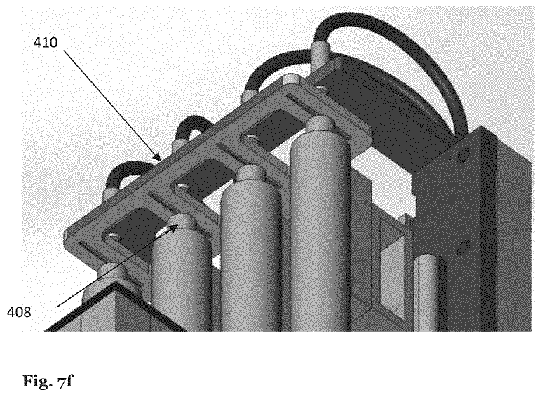

FIGS. 7a to 7f show schematic representations of parts of the automated liquid handling system according to a further embodiment of the present invention. The embodiment shown corresponds to the embodiment shown in FIGS. 4a-4j, wherein instead of the backwards positioned light sources (and the corresponding manifold means), the light sources are arranged such that the manifold means are supplied with the light beams from above. While this embodiment may not fully provide the specific advantages described earlier e.g. in connection with FIG. 4g, the present embodiment allows for other benefits: For example, it is possible to make use of a more simplified structure of the manifold means, as e.g. there is no need for two tunnels and light reflecting means, such as prisms. Moreover, more space on the backside of the manifold means may be available within the automated liquid handling system for other purposes. The illustrations of FIGS. 7a to 7f correspond to the illustrations of FIGS. 4a to 4f, wherein for the corresponding elements, the same reference numerals are used as in FIGS. 4a to 4f.

Further exemplary methods according to the present invention are described hereafter.

### Example 1: Processing of a PBMC Culture Obtained from Whole Blood and Imaging

A whole blood sample is received, e.g. about 1.5 to 2 ml whole blood, depending on the further processing.

The sample is transferred to a centrifuge tube, and the sample is fractionated by centrifugation at 150 g for 15 minutes, followed by centrifugation at 400 g for 20 minutes. The sample may be transferred by means of the automated transportation device of the automated liquid handling system.

The layer containing peripheral blood mononuclear cells (PBMC layer) is manually or preferably automatically detected by means of a camera module.

The PBMC layer, approximately 150 to 200 μl volume, is aspirated and transferred to a new tube. Aspiration and transfer may be performed by the automated transportation device of the automated liquid handling system.

About 1 ml of culture medium (e.g. RPMI-1640) is added to the new tube, e.g. from one of the storage containers comprised in the automated liquid handling system, and suspended.

Subsequently, the PBMC's are sedimented by means of centrifugation at 200 g for 10 minutes.

The supernatant is removed, e.g. by the automated transportation device, and 1 ml fresh culture medium (RPMI-1640) supplemented with 10% fetal calf serum is added to the tube and suspended.

About 50 μl of the resuspended PBMC-containing suspension is aspirated and transferred to a sample deposition well of a microscope sample carrier.

In this case, the automated transportation device is coupled to a pipette tip, which allows aspiration of the sample and its transfer into one or more sample deposition well of one or more microscope sample carriers.

Subsequently, the automated transportation device transports the microscope sample carrier(s), loaded with the sample, to the centrifuge. For transporting, the automated transportation device is directly coupled to the coupling section of the microscope sample carrier(s). The cells are sedimented by centrifuging at 200 g for 10 minutes.

The microscope sample carrier is then transported to a culturing position in an incubator which is kept at 37° C., 5% CO₂ for culturing the extracted cells.

### Example 2: Processing of a Bacterial Cell Culture and Imaging

In the following, a method for the extraction and culturing of bacterial cells comprised in circular immune cells (CICs) is described. CICs may engulf bacteria by phagocytosis, or alternative bacteria may be bound to CICs. The method can be fully automated by means of the automated liquid handling system.

At first, a whole blood sample is obtained.

The sample is transferred to a centrifuge tube, and the sample is fractionated by centrifugation at 150 g for 15 minutes, followed by centrifugation at 600 g for 20 minutes.

The layer containing circular immune cells (CIC layer) is manually or preferably automatically detected by means of a camera module.

The CIC layer, approximately 150 to 200 μl volume, is aspirated and transferred to a new tube.

About 1 ml of brain heart infusion broth (BHI broth) is added to the new tube and suspended.

In additional tubes, a bacterial titration standard is prepared by adding predetermined amounts of Staphylococcus aureus ATCC29213, and/or Escherichia coli ATCC25922 into 1 ml BHI broth, each.

From each tube, i.e. the tube comprising the resuspended CIC layer and the tubes comprising the bacterial cell standards, 50 μl are aspirated and placed into separate sample deposition wells of the microscope sample carrier.

The microscope sample carrier is then transported to a culturing position in an incubator of the automated liquid handling system, which is kept at 37° C. for 4-6 h for culturing the extracted bacterial cells and the cell standard.

Subsequently, the microscope sample carrier is transported to the centrifuge and centrifuged at 300 g for 15 to 20 minutes, such that the bacterial cells are smeared at the sample deposition surfaces.

The supernatants are removed, and the samples are dried in air.

A gram staining method is applied for the targeted bacteria.

After staining, the microscope sample carrier is transported to a mounting device for microscopic examination.

1.-37. (canceled)

38. Automated liquid handling system for processing a plurality of samples in at least one microscope sample carrier, wherein the microscope sample carrier comprises a plurality of sample deposition wells, wherein each sample deposition well is defined on its lateral sides by one or more lateral walls and on its bottom side by a sample deposition surface, the automated liquid handling system comprising:
a centrifuge adapted to centrifuge the microscope sample carrier;
an automated transportation device
adapted to transfer the plurality of samples and/or a plurality of liquids into and/or out of each of the plurality of sample deposition wells of the microscope sample carrier, and adapted for transporting the microscope sample carrier across the automated liquid handling system, wherein the automated transportation device is configured to couple with a coupling section of the microscope sample carrier; and
one or more storage containers for receiving and/or storing the plurality of samples and/or the plurality of liquids.

39. The automated liquid handling system claim 38, further comprising a first mounting device adapted to hold the at least one microscope sample carrier for the transfer of the plurality of samples and/or plurality of liquids into and/or out of each of the plurality of sample deposition wells by the automated transportation device; and/or further comprising a second mounting device adapted to hold the at least one microscope sample carrier for examination of the plurality of samples under a microscope, preferably further comprising a motorized microscope stage for holding the second mounting device during microscopic examination.

40. The automated liquid handling system of claim 38, further comprising a motorized microscope stage, the motorized microscope stage comprising one or more mounting sections adapted to hold the at least one microscope sample carrier for examination of the plurality of samples under a microscope.

41. The automated liquid handling system of claim 39, further comprising means for microscopically examining the at least one sample, preferably one or more inverted microscopes.

42. The automated liquid handling system of claim 39, wherein the first and/or second mounting device and/or mounting sections are adapted to hold a plurality of microscope sample carriers in parallel.

43. The automated liquid handling system of claim 38, further comprising: a microscope module which comprises a motorized microscope stage, and means for microscopically examining the at least one sample, preferably one or more inverted microscopes, wherein the microscope module is arranged in a fixed position within the automated liquid handling system; preferably wherein the automated transportation device is adapted for transporting the at least one microscope sample carrier across the automated liquid handling system in x, y and z direction, and adapted for transporting the at least one microscope sample carrier to the microscope module, wherein the microscope module physically decoupled from the automated transportation device.

44. The automated liquid handling system of claim 38, further comprising the at least one microscope sample carrier.

45. The automated liquid handling system of claim 38, wherein the plurality of sample deposition wells is arranged such that the sample deposition surfaces are in essentially one plane; and/or wherein the plurality of sample deposition wells is arranged in a regular pattern, such that the distance between neighboring sample deposition wells is constant.

46. The automated liquid handling system of claim 38, wherein the sample deposition surfaces are plane; and/or wherein each sample deposition well has a tapered shape towards the sample deposition surface.

47. The automated liquid handling system of claim 38, wherein the microscope sample carrier is partially or fully composed of an opaque material, preferably wherein the lateral walls of the microscope sample carrier are composed of an opaque plastic material; and/or wherein the sample deposition surfaces are composed of a transparent material, preferably a transparent plastic material.

48. The automated liquid handling system of claim 38, wherein each sample deposition surface has an area of between 0.5 mm² and 20 mm², preferably between 1 mm² and 15 mm², and more preferably between 6.6 mm² and 11.18 mm²; and/or wherein each sample deposition surface has a thickness of between 0.1 and 0.4 mm, preferably between 0.15 and 0.35 mm, further preferably of about 0.3 mm; and/or wherein each sample deposition surface has a thickness of between 0.17 and 0.25 mm, and/or wherein each sample deposition well has a volume of between 2 μl and 700 μ1, preferably between 5 μl and 500 μ1, more preferably between 20 μand 60 μ.

49. The automated liquid handling system of claim 38, wherein the sample deposition wells are arranged in one or more rows.

50. The automated liquid handling system of claim 38, wherein each sample deposition well is defined by an angle formed between the one or more lateral walls and the sample deposition surface, wherein the angle is between 70° and 110°, preferably between 80° and 100°, most preferably about 90°.

51. The automated liquid handling system of claim 38, wherein the automated transportation device comprises a robotic arm or robotic gripper for receiving one or more flanges or recesses of the coupling section of the microscope sample carrier.

52. A method for processing a plurality of samples in at least one microscope sample carrier, wherein the microscope sample carrier comprises a plurality of sample deposition wells, wherein each sample deposition well is defined on its lateral sides by one or more lateral walls and on its bottom side by a sample deposition surface, the method carried out by an automated liquid handling system, the method comprising:
applying, by an automated transportation device of the automated liquid handling system, each biological sample of a plurality of biological samples into at least one sample deposition well of the plurality of sample deposition wells;
separating, by a centrifuge of the automated liquid handling system, a plurality of first portions from a plurality of second portions of the plurality of the biological samples by means of application of a centrifugal force, wherein the plurality of first portions is deposited on the plurality of sample deposition surfaces; and
transporting, by the automated transportation device of the automated liquid handling system, the microscope sample carrier across the automated liquid handling system, wherein the automated transportation device is configured to couple with a coupling section of the microscope sample carrier.

53. The method of claim 52, wherein in the step of separating, the plurality of surfaces is in a position perpendicular to the axis of rotation; and/or wherein the plurality of first portions is deposited onto the plurality of sample deposition surfaces in uniform layers, preferably wherein the plurality of first portions comprises cells, which are deposited in uniform layers of single-cell thickness.

54. The method of claim 52, wherein one or more or all of the sample deposition surfaces and/or the inner surfaces of the lateral walls of the microscope sample carrier are prepared to specifically react with the plurality of first portions of the biological samples; and/or wherein one or more or all of the sample deposition surfaces is/are coated with adhesion promoters that increase the adhesion of biological cells to the surface.

55. The method of claim 52, further comprising at least one of the following steps, carried out by the automated liquid handling system:
fixing the deposited plurality of first portions;
staining the deposited, preferably fixed plurality of first portions;
washing the deposited, preferably fixed plurality of first portions;
drying the optionally stained or washed deposited plurality of first portions by removal of supernatants;
incubating, by means of an incubator, the plurality of samples and/or plurality of first portions at a predefined temperature and/or atmosphere for a predefined time interval; preferably wherein the step of drying comprises centrifuging the microscope sample carrier, preferably at a centrifugal force of 50 to 500 g and/or for a centrifugation time of between 0.5 and 5 min, and/or wherein the step of drying comprises aspirating supernatants from the microscope sample wells; and/or
wherein the method further comprises at least one of the following steps:
transporting, by the automated transportation device of the automated liquid handling system, the microscope sample carrier across the automated liquid handling system to a mounting device adapted to hold the at least one microscope sample carrier for examination of the plurality of biological samples and/or first portions under a microscope;
transporting, by the automated transportation device of the automated liquid handling system, the microscope sample carrier across the automated liquid handling system to a motorized microscope stage, the motorized microscope stage comprising one or more mounting sections adapted to hold the at least one microscope sample carrier for examination of the plurality of samples under a microscope; and
microscopically analyzing the plurality of biological samples and/or plurality of first portions.

56. The method of claim 52, wherein in the step of separating, the centrifuge of the automated liquid handling system is accelerated such that the uniform distribution of cells is not affected, by avoiding sudden acceleration motions.

57. A method for culturing biological cells in at least one microscope sample carrier, wherein the microscope sample carrier comprises a plurality of sample deposition wells, wherein each sample deposition well is defined on its lateral sides by one or more lateral walls and on its bottom side by a sample deposition surface, the method carried out by an automated liquid handling system, the method comprising:
applying, by an automated transportation device of the automated liquid handling system, each biological sample of a plurality of biological samples into at least one sample deposition well of the plurality of sample deposition wells; and
incubating, by an incubator of the automated liquid handling system, the plurality of biological samples.
